(19) United States
(12) Patent Application Publication
(10) Pub. No.: US 2023/0310198 A1
(43) Pub. Date: Oct. 5, 2023

(54) INTRAORAL DEVICES AND METHODS FOR MAKING AND USING SUCH DEVCICES

(71) Applicant: THE BOARD OF TRUSTEES OF THE LELAND STANFORD JUNIOR UNIVERSITY, Stanford, CA (US)

(72) Inventor: HyeRan Choo, Palo Alto, CA (US)

(21) Appl. No.: 18/206,517

(22) Filed: Jun. 6, 2023

Related U.S. Application Data
(63) Continuation of application No. PCT/US21/62982, filed on Dec. 11, 2021.
(60) Provisional application No. 63/124,626, filed on Dec. 11, 2020.

(51) Int. Cl. A61F 5/058; A61F 5/56

(52) U.S. Cl. CPC A61F 5/05891; A61F 5/566

(57) ABSTRACT

Devices and methods are provided for treating infant treating congenital oral defects or conditions, e.g., as an infant grows. In one example, an oral appliance is formed that includes a palatal plate sized for an individual infant, a curved pharyngeal extension extending from a posterior region of the palatal plate, and a screw mechanism that allows side portions of the palatal plate to move away from one another to increase a size of the device, which allows a single device to treat the infant as they grow. In another example, a series of oral appliances may be formed including a first appliance based on the individual infant's initial anatomy and one or more additional appliances sized based on predictive projection of growth of the infant.

### RELATED APPLICATION DATA

The present application is a continuation of co-pending International Application No. PCT/US2021/062982, filed Dec. 11, 2021, which claims benefit of U.S. provisional application Ser. No. 63/124,626, filed Dec. 11, 2020, the entire disclosures of which are expressly incorporated by reference herein.

### TECHNICAL FIELD

The present application relates to devices and methods for treating congenital oral defects or conditions, such as cleft lip and palate, Robin Sequence, or other craniofacial deformities, sleep apnea, and the like, and, more particularly, to intraoral devices for treating such defects or conditions in infants, and to methods for making and using such devices.

### BACKGROUND

Robin sequence is a combination of facial differences that are present when a baby is born and may include a small, underdeveloped lower jaw (“micrognathia”) and/or a tongue that is positioned further back in the mouth than normal (“glossoptosis”). Newborn infants with micrognathia and/or glossoptosis often require mandibular distraction osteogenesis surgery due to obstructive sleep apnea and/or feeding difficulties that they experience given their tongue's position and/or small jaw.

Alternatively, an oral appliance or palatal plate may be used to treat such conditions. To make such an oral appliance, a maxillary impression of the infant's oral anatomy is acquired, a plaster cast is formed based on the imprint, and an oral appliance is formed using the cast, e.g., from acrylic material, that includes a palatal plate and a pharyngeal extension (velar spur or tail). The palatal plate and pharyngeal extension may be inserted into the infant's mouth with the pharyngeal extension directed into the pharynx, e.g., descending down the vallecula epiglottica to anteriorly shift the base of the tongue and/or lower jaw and widen the infant's airway.

Because infants grow rapidly, an individual infant may quickly outgrow an oral appliance initially made for them. As a result, periodically, a practitioner may grind parameters of an individual palatal plate and/or pharyngeal extension in an attempt to accommodate the infant's growth and changing anatomy. Such modifications are only an estimate, however, with the practitioner typically visually guessing the modifications needed. Consequently, the practitioner may not make sufficient changes or may remove too much material, thereby rendering the oral appliance ineffective for further treatment and/or increasing discomfort to the infant as it is modified.

Alternatively, as an infant grows, a new imprint, cast, and oral appliance may be made periodically, but this requires the infant to repeatedly experience the procedure necessary to acquire each new imprint, which can be uncomfortable and/or distressing to the infant.

Therefore, devices and methods that improve treating congenital oral defects or conditions in infants would be useful.

### SUMMARY

The present application is directed to devices and methods for treating congenital oral defects or conditions, such as cleft lip and/or palate, Robin Sequence, or other orofacial or craniofacial deformities, feeding difficulties, breathing difficulties, sleep disorders, and the like. More particularly, intraoral devices are provided for treating congenital oral defects in neonatal infants, such as cleft lip and palate, Robin Sequence, or other craniofacial deformities, and/or other oral conditions, such as sleep apnea, e.g., due to micrognathia and/or glossoptosis, e.g., using a single adjustable oral appliance or a series of oral appliances. Methods for making and using such devices are also provided.

In accordance with one example, a method is provided for treating a congenital oral defect or condition, e.g., in a growing infant, using an oral appliance or orthodontic airway plate (“OAP”). Initially, an oral appliance is formed that includes a palatal plate including an anterior region and a posterior region opposite one another along an axis, a curved pharyngeal extension extending from the posterior region along the axis, and a screw mechanism. The oral appliance may be placed within the subject's oral cavity such that the pharyngeal extension directs the subject's tongue and/or lower jaw anteriorly.

After a desired treatment period, the palatal plate and pharyngeal extension may be split along the axis to create two palatal plate halves or side portions, and the screw mechanism actuated to direct the two palatal plate side portions away from one another to increase a size of the palatal plate and pharyngeal extension. Optionally, the gap between the side portions may then be filled. The oral appliance may then be placed within the subject's oral cavity for continued treatment. This process may be repeated multiple times, e.g., to accommodate an infant's growth without requiring a new oral appliance.

In accordance with another example, a device is provided for treating a congenital oral defect or condition that includes a palatal plate configured to be positioned within a subject's oral cavity and comprising an anterior region and a posterior region opposite one another and defining an axis therebetween; a curved pharyngeal extension extending from the posterior region along the axis configured to extend into the subject's pharynx to direct the subject's tongue and/or lower jaw anteriorly when the palatal plate is positioned within the oral cavity; and a screw mechanism coupled to the palatal plate, wherein the palatal plate is configured to be split along the axis to create two palatal plate side portions, and wherein the screw mechanism is actuatable to direct the two palatal plate side portions away from one another to increase a size of the palatal plate.

In accordance with another example, a device is provided for treating a congenital oral defect or condition that includes a palatal plate configured to be positioned within a subject's oral cavity and comprising an anterior region and a posterior region opposite one another and defining an axis therebetween; a curved pharyngeal extension extending from the posterior region along the axis configured to extend into the subject's pharynx to direct the subject's tongue and/or lower jaw anteriorly when the palatal plate is positioned within the oral cavity; a pair of anterior wires extending from the anterior region of the palatal plate for securing the device on the face relative to the oral cavity; and a screw mechanism coupled to the palatal plate, wherein the palatal plate is configured to be split along the axis to create two palatal plate side portions, and wherein the screw mechanism is actuatable to direct the two palatal plate side portions away from one another to increase a size of the palatal plate.

In accordance with still another example, a device is provided for treating an excessively wide palatal and/or facial cleft that includes a palatal plate configured to be positioned within a subject's oral cavity and comprising an anterior region and a posterior region opposite one another and defining an axis therebetween; a curved pharyngeal extension extending from the posterior region along the axis configured to extend into the subject's pharynx to direct the subject's tongue and/or lower jaw anteriorly when the palatal plate is positioned within the oral cavity; and a screw mechanism coupled to the palatal plate, wherein the palatal plate is configured to be split along the axis to create two palatal plate side portions with a gap between them, and wherein the screw mechanism is actuatable to direct the two palatal plate side portions towards one another to close the gap and decrease a size of the palatal plate.

In accordance with another example, a method is provided for making an oral appliance to treat a congenital oral defect or condition in an individual subject that includes acquiring one or both of a maxillary impression and an intraoral scan of the subject's oral cavity; based at least in part on one or both of the maxillary impression and intraoral scan, creating a cast including a cavity defining a palatal plate region and an extension region aligned along an axis; positioning a screw mechanism within the cavity; and introducing material into the cast to embed the screw mechanism within the material and form an oral appliance including a palatal plate and a curved pharyngeal extension extending from a posterior region of the palatal plate, wherein the palatal plate is configured to be split along the axis to create two palatal plate side portions, and wherein the screw mechanism is actuatable to direct the two palatal plate side portions away from one another to increase a size of the palatal plate.

In accordance with yet another example, a method is provided for making an oral appliance to treat a congenital oral defect or condition in an individual subject that includes acquiring imaging data regarding the anatomical configuration of the individual subject's oral, nasal, and pharyngeal cavities; and based at least in part on the imaging data, forming an oral appliance including a palatal plate comprising an anterior region and a posterior region opposite one another along an axis, and a curved pharyngeal extension extending from the posterior region along the axis with a screw mechanism embedded within the palatal plate, wherein the palatal plate is configured to be split along the axis to create two palatal plate side portions, and wherein the screw mechanism is actuatable to direct the two palatal plate side portions away from one another to increase a size of the palatal plate.

In accordance with still another example, a method is provided for making an oral appliance to treat a congenital oral defect or condition in an individual subject that includes acquiring imaging data regarding the anatomical configuration of the individual subject's oral, nasal, and pharyngeal cavities; and based at least in part on the imaging data, forming an oral appliance including a palatal plate and a screw mechanism embedded within the palatal plate; wherein the palatal plate is configured to be split along the axis to create two palatal plate side portions, and wherein the screw mechanism is actuatable to direct the two palatal plate side portions away from one another to increase a size of the palatal plate.

In accordance with yet another example, a method is provided for treating a congenital oral defect or condition that includes providing an oral appliance comprising a palatal plate comprising an anterior region and a posterior region opposite one another along an axis, a curved pharyngeal component extending from the posterior region along the axis, and a screw mechanism; after a desired treatment period, splitting the palatal plate and pharyngeal component along the axis to create two palatal plate side portions; repositioning the pharyngeal component, e.g., in a specific increment, periodically to gradually position the tongue more anteriorly, e.g., while maintaining the thickness of the pharyngeal component by adding the same type of rigid material anterior to the pharyngeal component and grinding the posterior surface of the pharyngeal component; and actuating the screw mechanism to direct the two palatal plate side portions away from one another to increase a size of the palatal plate. For certain conditions, it is desired to gradually reposition the pharyngeal component anteriorly, e.g., with specific interval (depending on the clinical situation), to stimulate more mandibular bone growth by promoting more anterior tongue movement as the patient grows. Gradual but more active anterior positioning of the tongue by the repositioning of the pharyngeal component promotes more expedited mandibular catch-up growth.

In accordance with still another example, a method is provided for treating a congenital oral defect or condition that includes providing an oral appliance comprising a palatal plate comprising an anterior region and a posterior region opposite one another along an axis, a curved pharyngeal extension extending from the posterior region along the axis, and a screw mechanism; after a desired treatment period, splitting the palatal plate and pharyngeal extension along the axis to create two palatal plate side portions; and actuating the screw mechanism to direct the two palatal plate side portions away from one another to increase a size of the palatal plate.

In accordance with another example, a method is provided for treating an excessively wide palatal and/or facial cleft that includes providing an oral appliance comprising a palatal plate comprising an anterior region and a posterior region opposite one another along an axis, a curved pharyngeal extension extending from the posterior region along the axis, and a screw mechanism; after a desired treatment period, splitting the palatal plate and pharyngeal extension along the axis to create two palatal plate side portions separated by a gap; and actuating the screw mechanism to direct the two palatal plate side portions towards one another to close the gap and decrease a size of the palatal plate.

In accordance with still another example, a method is provided for treating a congenital oral defect or condition that includes providing an oral appliance comprising a palatal plate comprising an anterior region and a posterior region opposite one another along an axis, a curved pharyngeal component extending from the posterior region along the axis, and a screw mechanism; positioning the oral appliance within a subject's oral cavity such that the pharyngeal component extends into the subject's pharynx to direct the subject's tongue and/or lower jaw anteriorly; after a desired treatment period, splitting the palatal plate and pharyngeal component along the axis to create two palatal plate side portions; actuating the screw mechanism to direct the two palatal plate side portions away from one another to increase a size of the palatal plate; repositioning the pharyngeal component in a specific increment periodically to gradually position the tongue more anteriorly while maintaining the thickness of the pharyngeal component by adding the same type of rigid material anterior to the pharyngeal component and grinding the posterior surface of the pharyngeal component; and positioning the increased-size oral appliance within the subject's oral cavity such that the pharyngeal extension extends into the subject's pharynx to direct the subject's tongue and/or lower jaw anteriorly.

In accordance with yet another example, a method is provided for treating a congenital oral defect or condition that includes providing an oral appliance comprising a palatal plate comprising an anterior region and a posterior region opposite one another along an axis, a curved pharyngeal extension extending from the posterior region along the axis, and a screw mechanism; positioning the oral appliance within a subject's oral cavity such that the pharyngeal extension extends into the subject's pharynx to direct the subject's tongue and/or lower jaw anteriorly; after a desired treatment period, splitting the palatal plate and pharyngeal extension along the axis to create two palatal plate side portions; actuating the screw mechanism to direct the two palatal plate side portions away from one another to increase a size of the palatal plate; and positioning the increased-size oral appliance within the subject's oral cavity such that the pharyngeal extension extends into the subject's pharynx to direct the subject's tongue and/or lower jaw anteriorly.

In accordance with another example, a method is provided for treating an excessively wide palatal and/or facial cleft that includes forming an oral appliance comprising a palatal plate comprising an anterior region and a posterior region opposite one another along an axis, a curved pharyngeal extension extending from the posterior region along the axis, and a screw mechanism; positioning the oral appliance within an infant's oral cavity such that the pharyngeal extension extends into the infant's pharynx to direct the infant's tongue and/or lower jaw anteriorly; after a desired treatment period, splitting the palatal plate and pharyngeal extension along the axis to create two palatal plate side portions; actuating the screw mechanism to direct the two palatal plate side portions towards one another to decrease a size of the palatal plate; and positioning the increased-size oral appliance within an infant's oral cavity such that the pharyngeal extension extends into the infant's pharynx to direct the infant's tongue and/or lower jaw anteriorly.

In accordance with still another example, a method is provided for treating an excessively wide palatal and/or facial cleft that includes forming an oral appliance comprising a palatal plate and a screw mechanism; positioning the oral appliance within an infant's oral cavity; after a desired treatment period, splitting the palatal plate and pharyngeal extension along the axis to create two palatal plate side portions; actuating the screw mechanism to direct the two palatal plate side portions towards one another to decrease a size of the palatal plate; and positioning the increased-size oral appliance within an infant's oral cavity such that the pharyngeal extension extends into the infant's pharynx to direct the infant's tongue and/or lower jaw anteriorly.

In accordance with another example, a method is provided for modifying an oral appliance to accommodate a growing infant that includes providing an oral appliance comprising a palatal plate comprising an anterior region and a posterior region opposite one another along an axis, a curved pharyngeal extension extending from the posterior region along the axis, and a screw mechanism; splitting the palatal plate and pharyngeal extension along the axis to create two palatal plate side portions; and actuating the screw mechanism to direct the two palatal plate side portions away from one another to increase a size of the palatal plate.

In accordance with yet another example, a method is provided for modifying an oral appliance to accommodate a growing infant that includes providing an oral appliance comprising a palatal plate comprising an anterior region and a posterior region opposite one another along an axis, a curved pharyngeal component extending from the posterior region along the axis, and a screw mechanism; splitting the palatal plate and pharyngeal component along the axis to create two palatal plate side portions; repositioning the pharyngeal component in a specific increment periodically to gradually position the tongue more anteriorly while maintaining the thickness of the pharyngeal component by adding the same type of rigid material anterior to the pharyngeal component and grinding the posterior surface of the pharyngeal component; and actuating the screw mechanism to direct the two palatal plate side portions away from one another to increase a size of the palatal plate.

In accordance with another example, a method is provided for making a set or series of sequential oral appliances to treat a congenital oral defect or condition in an individual subject, that includes acquiring imaging data regarding the anatomical configuration of the individual subject's oral cavity; based at least in part on the imaging data, forming an initial oral appliance including a palatal plate; using one or more processors to generate a model reflecting predicted changes in the anatomical configuration of the subject's oral cavity during a course of treatment for the congenital oral defect or condition; and based at least in part on the model, forming one or more additional oral appliances to replace the initial oral appliance at the onset of the treatment or during the course of treatment.

In accordance with another example, a set or series of sequential oral appliances is provided for treating a congenital oral defect or condition in an individual subject that includes an initial oral appliance including a palatal plate formed based on the anatomical configuration of the individual subject's oral cavity; and one or more additional oral appliances formed based at least in part on a model reflecting changes in the anatomical configuration of the subject's oral cavity at the onset of the treatment or during a course of treatment for the congenital oral defect or condition.

In accordance with still another example, a system is provide for making oral appliances to treat a congenital oral defect or condition in an individual subject that includes a 3D printer for creating individual oral appliances; memory for storing imaging data regarding the anatomical configuration of the individual subject's oral cavity; one or more processors operatively coupled to the printer and the memory and configured to, based at least in part on the imaging data, control the printer to form an initial oral appliance including a palatal plate; generate a model reflecting changes in the anatomical configuration of the subject's oral cavity during a course of treatment for the congenital oral defect or condition; and, based at least in part on the model, control the printer to form one or more additional oral appliances to replace the initial oral appliance during the course of treatment.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIGS. 1A and 1B are perspective and top views, respectively, of an oral appliance for treating an infant having a congenital oral defect and/or other condition.

FIGS. 2A-2C show an exemplary method for making the oral appliance of FIGS. 1A and 1B.

FIGS. 3A-3D show an exemplary method for increasing the size of the oral appliance of FIGS. 1A and 1B.

FIG. 4 is a cross-section of an infant's head showing placement of an oral appliance to direct the infant's tongue and/or lower jaw anteriorly.

FIG. 5 shows an exemplary method for making a series of oral appliances for treating an infant as the infant grows.

FIG. 6 is a schematic showing an exemplary system for creating a series of oral appliances for treating subjects having oral defects or other conditions.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

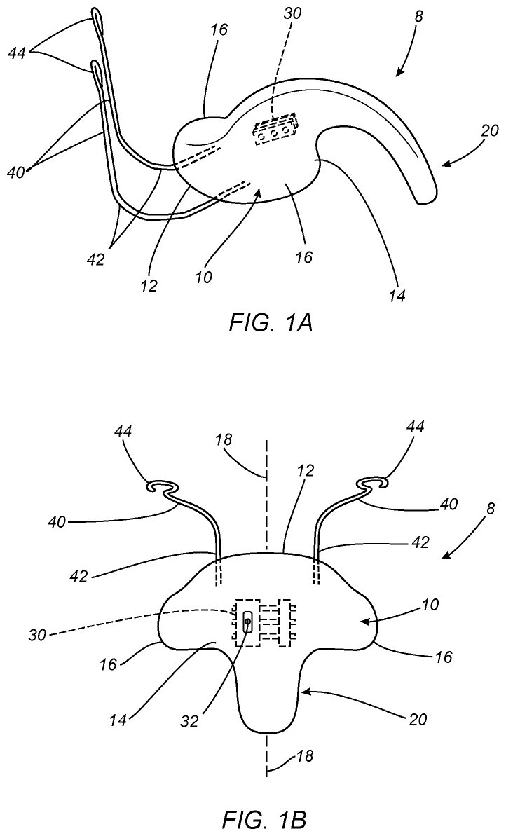

Turning to the drawings, FIGS. 1A and 1B show an example of a device 8, i.e., an oral appliance or orthodontic airway plate (“OAP”), for treating a growing infant with a congenital oral defect or condition, such as orofacial disfigurements related to cleft lip and/or palate, Robin Sequence, sleep apnea, e.g., due to micrognathia, retrognathia, glossoptosis, and the like, or other conditions. Although particularly useful for treating infants, the devices and methods herein may also be used to treat older children or even adults, e.g., suffering from sleep apnea or other oral conditions.

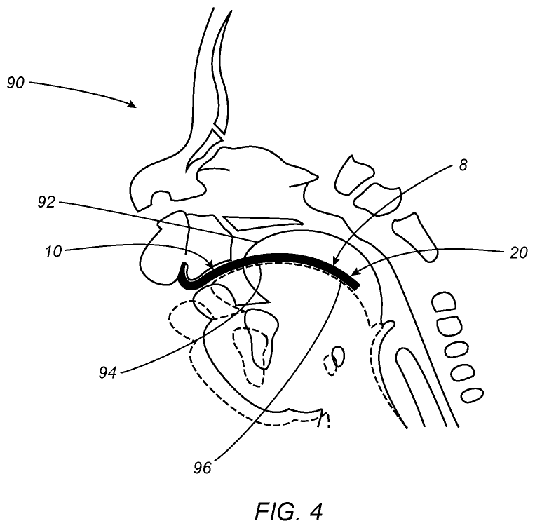

Generally, the oral appliance 8 includes a palatal plate 10 and pharyngeal extension 20 integrally molded or otherwise formed together, a screw mechanism 30 embedded or otherwise permanently attached to the palatal plate 12, and a pair of extension wires 40 extending from the palatal plate 10. The palatal plate 10 is sized and/or shaped to be positioned within a subject's oral cavity and includes an anterior region 12, a posterior region 14 opposite the anterior region 12, thereby defining an axis 18 therebetween, and lateral regions 16 on either side of the axis. The pharyngeal extension 20 extends from the posterior region 14 along the axis 18 that is configured to extend into the subject's pharynx to direct the subject's tongue and/or lower jaw anteriorly when the oral appliance 10 is positioned within the oral cavity, e.g., as shown in FIG. 4 and described further elsewhere herein. Optionally, for treating some conditions, the pharyngeal extension may not be needed and so may be omitted from the final oral appliance (not shown).

As can be seen in FIG. 1B, the screw mechanism 30 is at least partially embedded within the material of the palatal plate 10 such that an actuator 32 of the screw mechanism 30 remains exposed and/or otherwise accessible. In one example, the screw mechanism 30 is a jackscrew embedded generally centered within the palatal plate 10 that includes a pair of screw elements oriented substantially perpendicular to the axis 18. The actuator 32 may include a lead screw that may be rotated to cause the screw elements to move away from one another, i.e., substantially perpendicular to the axis 18.

The extension wires 40 may be attached to the anterior region 12 of the palatal plate 10, e.g., on either side of the axis 18. For example, first ends 42 of the wires 40 may be embedded in and/or otherwise permanently attached to the palatal plate 10, and second free ends 44 may be shaped to facilitate securing the oral appliance to the infant's face during use, as explained elsewhere herein. The wires 40 may be formed from substantially rigid and/or malleable material, e.g., stainless steel, aluminum, or other biocompatible material, as is known in the art.

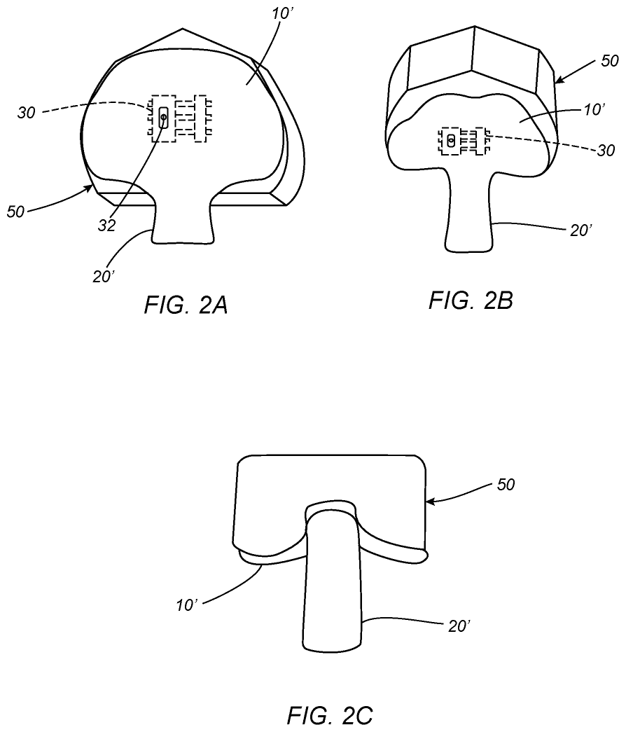

FIGS. 2A-2C show an exemplary method for initially making an oral appliance, such as the oral appliance 10. Initially, a maxillary impression may be acquired of the individual infant's oral cavity using conventional procedures. Based at least in part on the maxillary impression, a plaster cast or mold 50 may be formed including a cavity defining a palatal plate region. The screw mechanism 30 may be positioned at a desired location within the cavity, and acrylic or other biocompatible material 10′ may be introduced into the cast 50 to form the palatal plate 10, e.g., with a lower surface of the palatal plate 10 defining a size and/or shape based on the maxillary impression. An extension base 20′, e.g., formed from the same material as the palatal plate material 10′, may be positioned adjacent the cast 50 such that, when material is introduced into the cast 50, the material 10′ forms the palatal plate integral with the extension base 20′. The material may be cured or otherwise set using conventional methods, e.g., to provide a substantially rigid palatal plate 10. Exemplary materials for the palatal plate 10 may include acrylic or other biocompatible material, e.g., polymethyl methacrylate (PMMA) dispersed within an acrylic monomer or Lucitone Digital Print (LDP) material.

As can be seen in FIGS. 2A-2C, a standard extension base 20′ may be molded to the palatal plate material 10′ and then subsequently shaped, e.g., by grinding and/or otherwise removing material from the base to form an extension 20 having a desired shape for the individual infant. If necessary, the palatal plate 10′ material may also be further shaped, if desired to provide a desired shape and/or finish.

Alternatively, a cast or mold may be provided that may include a cavity defining both a palatal plate and extension region contiguous with one another (not shown), and the palatal plate 10 and pharyngeal extension 20 may be formed simultaneously when material is introduced into the cast. In a further alternative, the palatal plate 10 and pharyngeal extension 20 may be formed using other methods, e.g., using three-dimensional printing with the shape being programmed based at least in part on the maxillary impression.

The wires 40 may be attached to the palatal plate 10 after it is formed, e.g., by one or more of screwing or otherwise inserting the first ends 42 into the palatal plate 10, bonding with adhesive, heat fusing, and the like. Alternatively, the first ends 42 may be positioned within the cast and molded into the palatal plate 10 when the acrylic material is introduced.

Once the oral appliance 8 is ready, as shown in FIG. 4, the oral appliance 8 may be introduced into the oral cavity 92 of the infant 90 (or other subject), e.g., such that the palatal plate 10 is positioned above the infant's tongue 94 and the pharyngeal extension 20 extends into the infant's pharynx 96 e.g., descending down the vallecula epiglottica. The second ends 44 of the extension wires 40 may extend out of the infant's mouth and be taped or otherwise secured, e.g., to the infant's face using conventional methods to hold the oral appliance 8 in position while remaining within the infant's mouth. Given the shape and rigidity of the oral appliance, the pharyngeal extension 20 may apply an anterior force that, over time, directs the infant's tongue and/or lower jaw anteriorly.

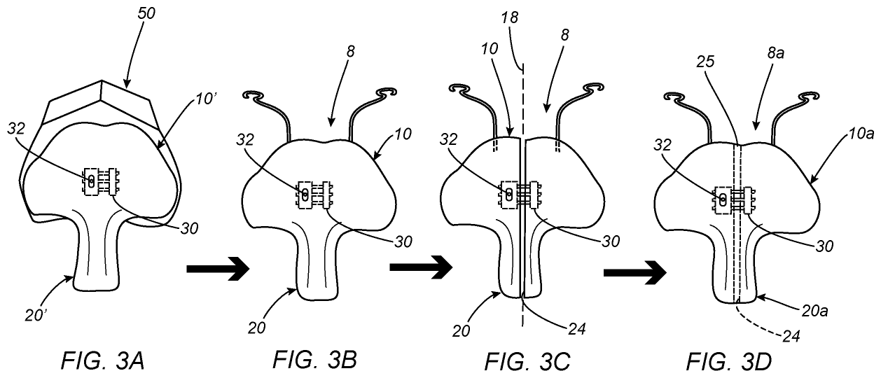

After a desired treatment period, e.g., three-to-four weeks, the oral appliance 8 may be modified to accommodate growth of the infant. For example, as shown in FIGS. 3A-3D, an initial oral appliance 8 (shown in FIGS. 3A and 3B) may be split and widened to increase the width of the palatal plate 10 and pharyngeal extension 20. As shown in FIG. 3C, the palatal plate 10 and pharyngeal extension 20 may be cut or otherwise split along the axis 18 to create two palatal plate side portions separated by a gap 24 but that remain connected together by the screw mechanism 30 embedded in the palatal plate 10. The screw mechanism 30 may then be actuated to direct the two palatal plate side portions away from one another to increase overall width of the palatal plate. For example, lead screw 32 may be rotated to direct the screw elements in the side portions away from one another substantially perpendicular to the axis 18.

The gap 24 may be increased a desired distance, e.g., about one or two millimeters (1-2 mm). Optionally, the gap 24 may be filled, e.g., by introducing acrylic or other material 25 into the gap 24 and curing or otherwise setting the material to provide an increased-size oral appliance 8a, e.g., as shown in FIG. 3D. If desired, the added material may be ground or otherwise modified to provide a continuous finished surface for the palatal plate 10a and pharyngeal extension 20a.

The increased-size oral appliance 8a may then be positioned within the infant's oral cavity 92 such that the pharyngeal extension 10a extends into the infant's pharynx 96, e.g., to continue to direct the infant's tongue and/or lower jaw anteriorly. This process may be repeated one or more times, as desired, e.g., over several weeks or months such that a single oral appliance may be used to treat the infant. In this manner, the infant may avoid having multiple maxillary impressions taken as they grow. For some conditions, it is desirable to gradually reposition the pharyngeal extension 20 anteriorly, e.g., periodically and/or at specific intervals (depending on the clinical situation), to stimulate mandibular bone growth by promoting anterior tongue movement as the infant grows. Gradual but more active anterior positioning of the tongue by the repositioning of the pharyngeal extension 20 may promote expedited mandibular catch-up growth.

In another example, an oral appliance such as the oral appliance 8 may be used to treat other conditions, such as Pierre Robin syndrome and/or other conditions that may involve an excessively wide palatal and/or facial cleft. Similar to other examples herein, the oral appliance 8 may be formed to include a palatal plate 10 with an embedded screw mechanism 30 and a curved pharyngeal extension 20 extending from a posterior region of the palatal plate 10. However, in this example, the screw mechanism 30 may be a constriction screw configured to direct opposite ends of the screw towards one another. For example, after a desired treatment period, the palatal plate 10 and pharyngeal extension 20 may be split along the axis to create two palatal plate side portions connected by the screw mechanism and separated by a gap. The screw mechanism 30 may then be actuated to direct the two palatal plate side portions towards one another, e.g., to close the gap and/or otherwise decrease a width or size of the palatal plate. The reduced-size oral appliance may be then positioned within an infant's oral cavity such that the pharyngeal extension extends into the infant's pharynx to continue to direct the infant's tongue and/or lower jaw anteriorly and/or reduce the width of the palatal and/or facial cleft.

In an alternative method, an oral appliance including a palatal plate and pharyngeal extension may be modeled and formed using three-dimensional printing based on the initial maxillary impression of the infant's oral cavity without a screw mechanism. Rather than splitting the oral appliance, a new model may be generated based simply on increasing the width of the palatal plate and pharyngeal extension, i.e., simulating splitting the oral appliance and then increasing a filling the created gap, and then a new oral appliance may be formed by three-dimensional printing without a new maxillary impression. Optionally, for treating some conditions, the pharyngeal extension may not be needed and so the final oral appliance may include a palatal plate without a pharyngeal extension.

Alternatively, all of the components of the oral appliance, e.g., the anterior extension wires, palatal plate, and pharyngeal extension may be printed as a one-piece, integrated assembly, e.g., from the same material. Thus, as an infant or other young patient grows and needs a larger oral appliance, a virtually saved initial oral appliance may be enlarged, e.g., maintaining the size and/or angulation of the pharyngeal extension to treat the subject, but increasing the size of the palatal plate based on their growth.

Optionally, a plurality of standard size oral appliances may be printed and formed, e.g., having a base design palatal plate but with different length, angulation, and/or width of the pharyngeal extension and/or with different sizes of anterior extension wires. For example, a set of different combinations may be provided to a practitioner, who may select one that fits for an individual patient.

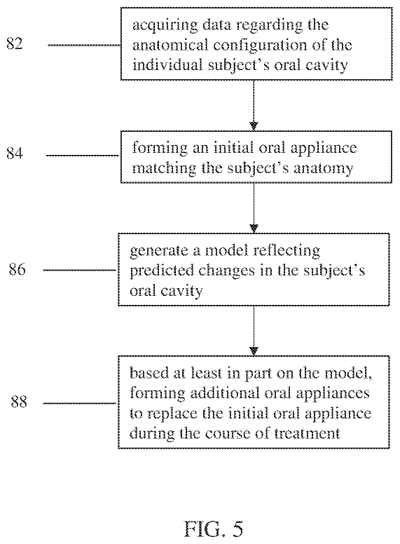

Turning to FIG. 5, an exemplary method is shown for making a custom set of oral appliances to treat a congenital oral defect or condition in an individual subject, e.g., an neonatal infant. Initially, at step 82, data may be acquired regarding the anatomical configuration of the individual infant's oral cavity. For example, a maxillary impression of the infant's oral cavity may be acquired, e.g., using conventional procedures, such as those described elsewhere herein. In addition or alternatively, other methods may be used to identify the configuration of the individual infant's oral cavity, e.g., external imaging such as computer tomography (CT) scan, magnetic resonance imaging (MRI), intraoral scanner, and the like, or merging of such imaging data to form a digital or physical duplicate of the oral and pharyngeal cavities of the subject. For example, a plurality of anatomical images may be acquired using such imaging methods, which may be combined digitally to generate a composite anatomic model of the subject's individual anatomy.

At step 84, an initial oral appliance may then be formed based on the actual anatomical configuration of the individual infant. For example, an oral appliance may be formed similar to the oral appliance 8 shown in FIGS. 1A and 1B, e.g., including a palatal plate, a curved pharyngeal extension, and a pair of extension wires, but without a screw mechanism. In one method, the palatal plate and pharyngeal extension may be integrally formed by deposition or other 3D printing material into the shape of the desired oral appliance, e.g., using acrylic or other biocompatible material, such as those described elsewhere herein. Alternatively, the oral appliance may be formed from casting, molding, and the like, e.g., similar to the methods described elsewhere herein. The extension wires may be attached to the resulting palatal plate or, alternatively, may also be formed during the 3D printing integrally with the palatal plate. Optionally, similar to other devices and methods herein, for treating some conditions, the pharyngeal extension may not be needed and so may be omitted from the oral appliance (not shown).

At step 86, a model may be generated reflecting predicted changes in the anatomical configuration of the infant's oral cavity during a course of treatment. For example, the model may predict changes in the infant's anatomy based on one or both of anticipated growth of the infant and anatomical changes resulting from use of an oral appliance during the course of treatment. In one example, as described further elsewhere herein, the model may initially begin with a virtual oral appliance having dimensions based on the actual initial oral appliance, i.e., thereby corresponding to the infant's actual initial anatomy, and then project changes, e.g., based on comparisons to actual changes in other infants undergoing similar treatments or other data. For example, a software algorithm may generate a virtual model of the initial oral appliance and then generated projected changes at desired intervals and modify the virtual model to reflect the projected changes in the infant's anatomy. For example, the algorithm may incrementally increase or narrow a width of the palatal plate of each of the additional oral appliances compared to the width of the initial oral appliance (depending on the condition being treated) In addition or alternatively, if the oral appliance includes a pharyngeal extension, the algorithm may also incrementally move the pharyngeal extension of each of the additional oral appliances anteriorly compared to the initial oral appliance. For example, it may be desirable to gradually reposition the pharyngeal extension anteriorly in each subsequent oral appliance, e.g., periodically and/or at specific intervals (depending on the clinical situation), to stimulate mandibular bone growth by promoting anterior tongue movement as the infant grows. Gradual but more active anterior positioning of the tongue by the repositioning of the pharyngeal extension may promote expedited mandibular catch-up growth.

Finally, at step 88, one or more additional oral appliances may be created, e.g., 3D printed or otherwise formed, based on the projected virtual model, e.g., to replace the initial oral appliance during the course of treatment. For example, a plurality of additional devices may be formed that increase in size and/or otherwise change dimensions based on the virtual model, e.g., based on desired periodic or desired time intervals during an anticipated course of treatment. The resulting set of oral appliances may then be provided to a doctor or other medical practitioner and/or caregiver, who may use the set to treat the individual infant. For example, the initial oral appliance may be used for an initial time period, and then, the additional oral appliances may be used sequentially after each time interval used by the model as the basis for each additional oral appliance.

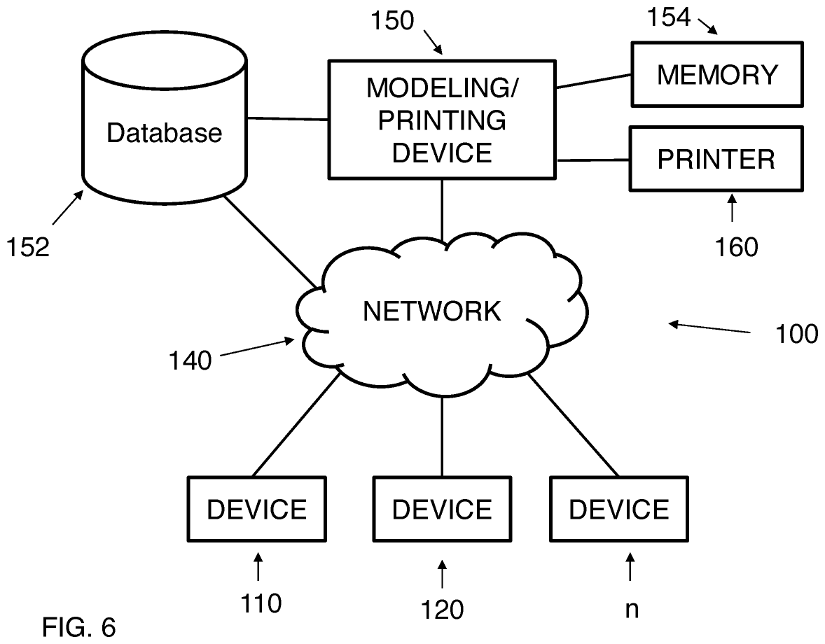

Turning to FIG. 6, an exemplary system 100 is shown that may be used to scale creating sets of custom oral appliances for use by multiple users, e.g., doctors or other medical practitioners treating different individual infants at one or more locations, e.g., which may be remote from one another and/or from the maker of the oral appliances. Generally, the system 8 includes various devices connected to a network 140, such as user devices 110, 120, n, and a modeling/printing device 150. The network 140 may be a private or public network, including a wide area network (“WAN”), a local area network (“LAN”), an intranet, a wireless network, a short messaging service (“SMS”), and/or a telephony network. For example, any such network may incorporate several different types of networks including a WAN, a LAN, and/or a wireless network. One such network including multiple different types of networks is the Internet, e.g., which may be used if the user devices 110, 120, n and modeling/printing device 150 are located remote from one another. Alternatively, the network 140 may simply be a local and/or private network, e.g., if the users are all at the same physical location.

The modeling/printing device 150 may include one or more processors (not shown) that are operatively coupled to a database 152, which may be used to generate virtual models, memory 154 for storing anatomical data regarding individual subjects, and one or more printers (one printer 160 shown), e.g., 3D printers for forming sets of custom oral appliances, such as those described above. For example, the modeling/printing device 150 may include one or more computer systems, e.g., servers, communicating with one or more databases, e.g., including one or more processors, memory and/or storage devices, and communication interfaces for communicating via the network 140. The device 150 may include one or more hardware-based components and/or software-based modules for performing the various functions related to the methods performed, as described elsewhere herein. Although only one device 150 is shown, it will be appreciated that multiple servers or other device (not shown) may be provided at the same or different locations that operate cooperatively to perform the functions described herein. Thus, all of the components cooperating with the device 150 may be located at the same physical location or may be distributed, e.g., located at different physical locations and communicating with one another via the network 140.

During use, for example, the maker of the oral appliances may receive requests or orders from the user devices 110, 120, n, e.g., from individual doctors, including anatomical data related to individual subjects being treated, which may be processed by the processor 150 to create corresponding sets of oral appliances, which may then be shipped or otherwise provided to the respective users for use with their patients.

For example, each user 110, 120, n may acquire the anatomical data for the infant(s) they are treating, e.g., by acquiring maxillary impressions and/or other images, and communicate the resulting raw images and/or processed data to the modeling/printing device 150 via a network 140, which may be stored in memory 154.

The modeling/printing device 150 may then use the data along with information from the database 152 to generate virtual models and corresponding sets of oral appliances, e.g., as described above. In one example, the database 152 may include historical data obtained from studying prior subjects treated with oral appliances and/or analyzing oral appliances used to treat subjects, such as the adjustable oral appliances described elsewhere herein or conventional oral appliances that are used and/or modified by users manually. The database 152 may be updated as additional data is acquired, which may be used by a software algorithm of the device 150 when it models and creates new sets of oral appliances.

In one example, maxillary arch models may be generated based on actual treatments of subjects, e.g., by acquiring anatomical data of the subjects' oral activities and/or dimensions of the actual oral appliances used, multiple times during the treatments. Such models may then be used in a superimposition method to predict and create new sets of oral appliances for new subjects.

In describing representative examples, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for treating a congenital oral defect or condition, comprising:
a palatal plate configured to be positioned within a subject's oral cavity and comprising an anterior region and a posterior region opposite one another and defining an axis therebetween;
a curved pharyngeal extension extending from the posterior region along the axis configured to extend into the subject's pharynx to direct the subject's tongue and/or lower jaw anteriorly when the palatal plate is positioned within the oral cavity; and
a screw mechanism coupled to the palatal plate,
wherein the palatal plate is configured to be split along the axis to create two palatal plate side portions, and wherein the screw mechanism is actuatable to direct the two palatal plate side portions away from one another to increase a size of the palatal plate or to direct the two palatal plate side portions towards one another to close the gap and decrease a size of the palatal plate.

2. The device of claim 1, further comprising one or more anterior wires extending from the anterior region of the palatal plate for securing the device on the face relative to the oral cavity.

3-5. (canceled)

6. The device of claim 1, wherein the screw mechanism is at least partially embedded within material of the palatal plate such that an actuator of the screw mechanism is exposed.

7. The device of claim 6, wherein the screw mechanism comprises a jackscrew embedded in the material of the palatal plate and wherein the actuator comprises a lead screw, the jackscrew defining a screw axis orthogonal to the axis of the palatal plate.

8. The device of claim 1, wherein the palatal plate is formed from acrylic resin.

9. (canceled)

10. A method for making an oral appliance to treat a congenital oral defect or condition in an individual subject, comprising:
acquiring imaging data regarding the anatomical configuration of the individual subject's oral, nasal, and pharyngeal cavities; and
based at least in part on the imaging data, forming an oral appliance including a palatal plate comprising an anterior region and a posterior region opposite one another along an axis, and a curved pharyngeal extension extending from the posterior region along the axis with a screw mechanism embedded within the palatal plate,
wherein the palatal plate is configured to be split along the axis to create two palatal plate side portions, and wherein the screw mechanism is actuatable to direct the two palatal plate side portions away from one another to increase a size of the palatal plate.

11. (canceled)

12. The method of claim 10, wherein acquiring the imaging data comprises acquiring one or more images using one or more of an intraoral scan, CT scan, and MRI, or a combination of such images to generate a composite anatomic model.

13. (canceled)

14. A method for treating a congenital oral defect or condition, the method comprising:
providing an oral appliance comprising a palatal plate comprising an anterior region and a posterior region opposite one another along an axis, a curved pharyngeal extension extending from the posterior region along the axis, and a screw mechanism;
after a desired treatment period, splitting the palatal plate and pharyngeal extension along the axis to create two palatal plate side portions; and
actuating the screw mechanism to direct the two palatal plate side portions away from one another to increase a size of the palatal plate.

15. The method of claim 14, further comprising repositioning the pharyngeal extension in a specific increment periodically to gradually position the infant's tongue more anteriorly.

16. The method of claim 15, wherein repositioning the pharyngeal extension comprises:
adding material to the oral appliance anterior to the pharyngeal extension; and
grinding the posterior surface of the pharyngeal extension.

17. The method of claim 16, wherein material is added to the oral appliance to maintain the thickness of the pharyngeal extension.

18. The method of claim 16, wherein the added material is the same as the original pharyngeal extension.

19. The method of claim 14, further comprising filling a gap between the side portions with filler material.

20. The method of claim 19, wherein the palatal plate and pharyngeal extension are integrally formed from rigid material, and wherein the filler material is the same type of rigid material.

21. The method of claim 14, further comprising:
positioning the oral appliance within a subject's oral cavity such that the pharyngeal extension extends into the subject's pharynx to direct the subject's tongue and/or lower jaw anteriorly before splitting the palatal plate and pharyngeal extension; and
removing the oral appliance from the subject's oral cavity,
wherein splitting the palatal plate and pharyngeal extension and actuating the screw mechanism are performed after removing the oral appliance.

22. The method of claim 21, further comprising positioning the increased-size oral appliance within the subject's oral cavity such that the pharyngeal extension extends into the subject's pharynx to direct the subject's tongue and/or lower jaw anteriorly.

23. (canceled)

24. The method of claim 14, wherein providing the oral appliance comprises:
acquiring one or both of a maxillary impression and an intraoral scan of the subject's oral cavity;
based at least in part on one or both of the maxillary impression and intraoral scan, creating a cast including a cavity defining a palatal plate region and an extension region;
introducing material into the cast to form the oral appliance.

25-38. (canceled)

39. The method of claim 14, wherein the subject is an infant.

40. The method of claim 14, wherein the oral appliance is used to treat an infant with one or more of cleft lip and palate, Robin Sequence, sleep apnea, or sleep disordered breathing.

41-55. (canceled)

56. A method for making a set or series of sequential oral appliances to treat a congenital oral defect or condition in an individual subject, comprising:
acquiring imaging data regarding the anatomical configuration of the individual subject's oral cavity;
based at least in part on the imaging data, forming an initial oral appliance including a palatal plate;
using one or more processors to generate a model reflecting predicted changes in the anatomical configuration of the subject's oral cavity during a course of treatment for the congenital oral defect or condition; and
based at least in part on the model, forming one or more additional oral appliances to replace the initial oral appliance at the onset of the treatment or during the course of treatment.

57-73. (canceled)
